(19) United States
(12) Patent Application Publication
(10) Pub. No.: US 2021/0016884 A1
(43) Pub. Date: Jan. 21, 2021

(54) AERIAL DRONE AIR TREATING DEVICE AND METHOD OF TREATING AIR THEREWITH

(71) Applicant: The Procter & Gamble Company, Cincinnati, OH (US)

(72) Inventors: Nicola John POLICICCHIO, Mason, OH (US); Dana Paul GRUENBACHER, Fairfield, OH (US); Todd Michael WERNICKE, Cincinnati, OH (US); Glenn Allen BRADBURY, Mason, OH (US)

(21) Appl. No.: 17/061,603

(22) Filed: Oct. 2, 2020

Related U.S. Application Data
(63) Continuation of application No. 15/369,914, filed on Dec. 6, 2016, now abandoned.

(51) Int. Cl. B64D 1/18; A61L 9/14; B64C 39/02

(52) U.S. Cl. CPC B64D 1/18; A61L 9/14; B64C 39/024; A61L 2209/14; A61L 9/125; B64C 2201/141; B64C 2201/12; B64C 2201/108; A61L 2209/111

(57) ABSTRACT

A drone which can be piloted autonomously or by a user. The drone has an air treatment dispenser for spraying, or otherwise treating, a target area with one or more desired air treatments. The drone may operate as deemed desirable by a user, upon a predetermined schedule or in response to a demand signal from one or more target areas. By delivering the air treatments from an elevated position, the target area can be more uniformly and efficiently treated.

### FIELD OF THE INVENTION

The present invention relates to a controllable drone having the ability to treat air from various elevations.

### BACKGROUND OF THE INVENTION

Air fresheners and other air treatment dispensers are well known in the art. For example, a volatile composition dispenser may be configured for use as an energized dispenser (i.e. powered by electricity; or chemical reactions, such as catalyst fuel systems; or solar powered; or the like). Exemplary energized volatile composition dispensers often have a powered delivery which may include a heating element, a piezo element, thermal ink jet element and/or fan assembly, or the like. More particularly, the volatile composition dispenser may be an electrical volatile composition dispenser, a non-limiting example of an electrical wall-plug volatile composition dispenser is described in U.S. Pat. No. 7,223,361; or may be a battery (including rechargeable) powered volatile composition dispenser having a heating and/or fan element. The volatile material may be formulated to optimally diffuse upon delivery.

The volatile composition dispenser may utilize a “drop-on-demand” ink-jet type printing process where a fluid ink is forced under pressure through a very small orifice of a diameter typically about 0.0024 inches (5-50 microns) in the form of minute droplets by rapid pressure impulses. The rapid pressure impulses are typically generated in the print head by either expansion of a piezoelectric crystal vibrating at a high frequency or volatilization of a volatile composition (e.g. solvent, water, propellant) within the ink by rapid heating cycles. The piezoelectric crystal expansion causes the volatile composition to pass through the orifice as minute droplets in proportion to the number of crystal vibrations. Thermal ink jet printers employ a heating element within the print head to volatilize a portion of the composition.

But such air treatment dispensers may not be suitable for all users. The user may be of limited mobility or not have the dexterity to manipulate the device and reach the areas desired be treated. An elderly user may tire before the task is completed. Treatment according to the prior art may be infeasible for such a user.

Furthermore, not all air treatment tasks optimally occur at floor level. A volatile composition dispenser used at floor level may not adequately disperse the composition. The composition may drop to the floor and not be entrained in ambient air currents for dispersal. Volatile compositions dispensed at higher elevations have greater residence time in the air and can be better dispersed and volatilized than like compositions dispensed at lower elevations.

Or the user may not have the dexterity needed to complete the task. Or areas sought to be treated may be out of reach. For example, the area to be treated may be too high or obscured by other objects. Treatment according to the prior art may be infeasible for such a user or for areas disposed out of reach.

Accordingly a new approach is needed, which provides for users of limited mobility/dexterity the capability to adequately perform air treatment and/or climate control. Likewise, a new approach is needed to treat elevated areas out of reach of healthy persons and to treat areas which cannot be reached by persons of limited mobility/dexterity.

The invention proposes to use aerial drones to assist in certain air treatment and/or climate control tasks. Various drones are discussed in U.S. Pat. Nos. 8,214,088; 8,474,761; 8,498,447; 8,594,862; 8,662,438; 8,725,314; 8,958,928; 8,983,684; 8,989,924; 2015/0120094 and 2015/035437. Aerial drones have commonly been used for hobby purposes. Drones may also be used for utilitarian purposes. For example, drones may be used to monitor power lines, as discussed in U.S. Pat. No. 4,818,990 or to deliver mail, as discussed in US 2014/0254896. Drones have been proposed to clean outdoor solar panels using brooms. US 2015/0274294 teaches the use of an aerial vehicle which sprays water/soap onto a window. But 2015/0274294 does not teach how to use the drone's elevation to disperse an air treatment composition. The present invention overcomes the problems of users not being able to employ conventional air treatment dispensers for certain air treatment tasks.

### SUMMARY OF THE INVENTION

In one embodiment the invention comprises a drone suitable for air treatment tasks. The drone comprises a chassis for holding components of the drone, such as at least one rotary wing driven by a respective motor and an air treatment dispenser. The drone may optionally be piloted by a controller for piloting the drone at speed and altitude relative to an area to be treated or autonomously perform air treatment on demand or on a preset schedule. In another embodiment the invention comprises a method of treating air at a desired elevation using a drone.

### BRIEF DESCRIPTION OF THE DRAWINGS

FIG. 1A is a diagrammatic view of a piloting device and a drone suitable for being controlled in accordance with the invention, the drone having asymmetric air treatment dispensers disposed on the head thereof.

FIG. 1B is a diagrammatic view of a piloting device and a drone suitable for being controlled in accordance with the invention, the drone having two air treatment dispensers, one air treatment dispenser configured to spray upwardly and one air treatment dispenser configured to spray downwardly.

FIG. 2 is a schematic view of an environment having a drone with an air freshener responsive to local sensors.

FIG. 3 is a diagrammatic view of a piloting device and a drone suitable for being controlled in accordance with the invention, the left side of the drone having two air treatment filters on the exhaust of the rotary wings, the right side of the drone have two air treatment filters on the intake to the rotary wings.

### DETAILED DESCRIPTION OF THE INVENTION

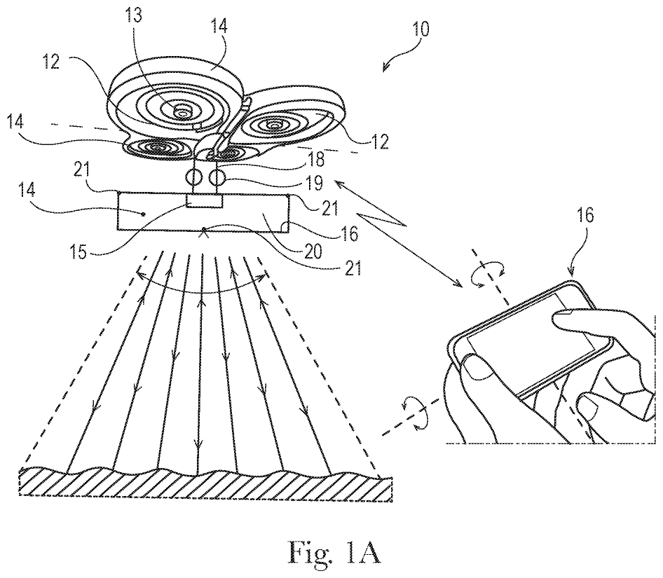

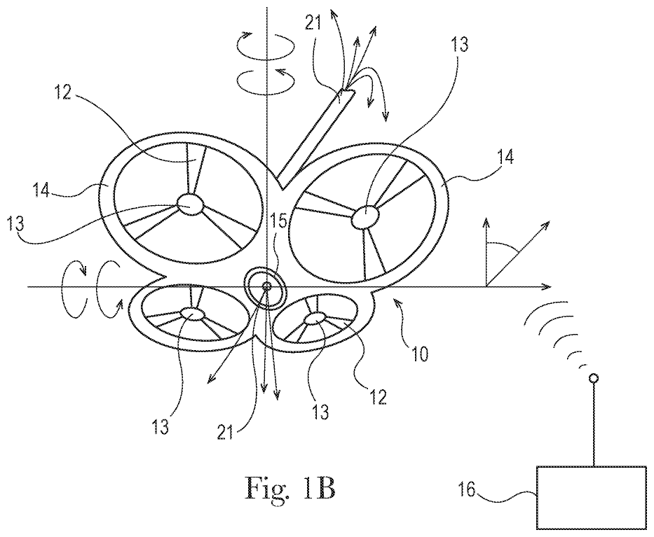

Referring to FIGS. 1A and 1B, the invention comprises a drone (10) suitable for dispensing an air treatment while flying. The drone (10) has an air treatment dispenser (21) mounted thereon.

Particularly, a drone (10) is a flying machine which is remotely piloted by means of a control device. A drone (10) according to the present invention may be fixed wing, dragonfly flappable wing or, more likely, rotary wing (12), comprising one or more rotors held in position by a chassis (14). The rotors may be driven by independently controllable respective motors (13) so the drone (10) can be controlled in attitude, speed and ground position. Each motor (13) may be controlled by a respective microcontroller. The set of microcontrollers may be controlled by a central controller (15). The central controller (15) may also control actuation of the air treatment dispenser upon demand from a user, according to schedule and/or and determined by autonomously by one or more sensors. A drone (10) having four such motors (13) and four respective controllers is commonly referred to as a quadricopter.

The rotary wing (12) may define a plane. Components of the drone (10) may be referenced to this plane. For example, a head (20) for an air treatment dispenser (21) may be disposed below this plane, while a reservoir for receiving air treatment may be disposed below, coplanar with, parallel to or above this plane.

An asynchronous serial communication link may be provided between the central controller (15) and each microcontroller. The central controller (15) may send simultaneous, synchronous control messages with at least one instruction specified by an address parameter of a destination microcontroller. The destination microcontroller extracts and executes the respective instruction. The control messages may be synchronized and executed as described in U.S. Pat. No. 8,725,314 assigned to Parrot of Paris France and incorporated herein by reference.

The drone (10) may be controlled through signals emitted by a tilt sensor, with the sensor moves being replicated by the drone (10), so that the drone (10) may tilt about a desired pitch axis relative to the horizontal plane. Other commands may include climb/descent from a throttle control and right/left rotation about a yaw axis.

The drone (10) may measure altitude, i.e. instantaneous position in the vertical direction relative to a fixed system, such as a Galilean system. Altitude may be measured by an ultrasound telemeter, commonly referred to as an altimeter, and comprising an acoustic transducer which remits/receives ultrasounds. The time separating emission and reception is proportionate to and allows determination of the distance to a target surface. A typical transducer emits echoes in a 55 degree cone aperture at a frequency of 25 Hz. A barometric pressure sensor may be used to measure variations in atmospheric pressure in flight. The pressure corresponds to altitude. In some embodiments, the drone (10) may include a radar and/or lidar. The radar can be used to determine the range, altitude, direction or speed of objects. Lidar measures distance by illuminating a target with a laser and analyzing the reflected light, thus providing accurate 3D mapping of the environment and specific objects.

Multiple telemeter and pressure sensors may be used in conjunction to control the attitude as described in U.S. Pat. No. 8,989,924 assigned to Parrot of Paris, France and incorporated herein by reference. If desired, a microwave radar system may be used to avoid humans, pets and other objects.

An altitude of zero may correspond to a reference position on the floor, a tabletop, countertop or other target surface. Knowing the difference in height between various target surfaces (40), one of ordinary skill can advantageously adapt the drone (10) of the present invention to clean floors and various other surfaces at different elevations and treat the ambient at the same or yet other elevations.

The drone (10) may be piloted by a piloting device (16), including but not limited to a phone or a multimedia player having a touch screen and an integrated accelerometer, for example a cellular phone of the iPhone type or a multimedia player or tablet of the iPod Touch or iPad type (registered trademarks of Apple Inc., USA), as are known in the art. Communication with the drone (10) may occur from the phone, tablet or player via a wired or wireless connection as are known in the art. Suitable wireless connections include WiFi IEEE 802.11 and Bluetooth (registered trademark). Control signals received by the drone (10) may articulate the drone (10) about the pitch and roll axes using a local reference frame relative to an absolute reference frame to determine angular orientation. A suitable pitch/roll control scheme is described in U.S. Pat. No. 8,594,862 assigned to Parrot of Paris, France and incorporated herein by reference.

Proprioceptive sensors used with the drone (10) can sense position, orientation and speed of the drone (10). For example, accelerometers are used to measure acceleration, from which velocity can be calculated by integration; tilt sensors to measure inclination; force sensors to measure contact force with environment; position sensors indicate the actual position of the drone (10), from which the velocity can be calculated by derivation. Information about contact force with a target surface and drone (10) current/desired motion to maintain dynamic balance is useful for completing the cleaning task described herein. The drone (10) may be equipped with one or more accelerometers, three-axis gyrometers and/or altimeters.

Arrays of tactels can be used to receive data on objects touched by the drone (10). The arrays of tactels, i.e. tactile sensors, provide information about forces and torques transferred between the drone (10) and the objects, particularly a target surface optionally being cleaned. The tactels allow the user to know whether or not the correct force is being applied to the target surface for efficacious cleaning. The drone (10) may be moved from position to position, as described in 2015/0120094, incorporated herein by reference.

To produce an image of the surroundings, the drone (10) can have vision sensors, such as charge-coupled device (CCD) cameras. Sound sensors (e.g. microphones) may optionally allow the drone (10) to hear speech and environmental sounds, recognize objects and determine properties of the objects. Ultrasonic sensors measure speed and distances to surrounding objects. Vision sensors enable omnidirectional imaging (360-degree) high definition video capture. Infrared sensors provide thermal imaging. The drone (10) may particularly be equipped with one or more cameras, such as a vertical view and/or front view camera.

The drone (10) may be capable of hovering under autopilot. The drone (10) may use a vertically oriented camera to continuously update a multi-representation of a pyramid of images. For each image an iterative optical flow algorithm provides a texturing parameter representative of the micro-contrasts in the picked-up scene and an approximation of the speed. If predetermined criteria are satisfied, the drone (10) may switch from the optical-flow algorithm to a corner detector algorithm. This process allows the drone (10) to hover for cleaning as described herein. Suitable drones (10), methods of piloting and methods for evaluating horizontal speed to allow cleaning are described in U.S. Pat. Nos. 8,474,761 and 8,498,447, both assigned to Parrot of Paris, France and incorporated herein by reference. The drone (10) may have a self-contained stabilizer system as described in U.S. Pat. No. 8,214,088 assigned to Parrot of Paris, France and incorporated herein by reference.

Motion of the drone (10) can be realized by actuators. The actuators can include electric, pneumatic, hydraulic, piezoelectric, ultrasonic, and other actuators. Hydraulic and electric actuators have a very rigid behavior and can only be made to act in a compliant manner through the use of relatively complex feedback control strategies. While electric coreless motor actuators are better suited for high speed and low load applications, hydraulic ones operate well at low speed and high load applications.

Piezoelectric actuators generate a small movement with a high force capability when voltage is applied. They can be used for ultra-precise positioning and for generating and handling high forces or pressures in static or dynamic situations.

Ultrasonic actuators are designed to produce movements in micrometer order at ultrasonic frequencies (over 20 kHz). They are useful for controlling vibration, positioning applications and quick switching.

Pneumatic actuators operate on the basis of gas compressibility. As they are inflated, they expand along the axis, and as they deflate, they contract. If one end is fixed, the other will move in a linear trajectory. These actuators are intended for low speed and low/medium load applications. Between pneumatic actuators there are: cylinders, bellows, pneumatic engines, pneumatic stepper motors and pneumatic artificial muscles.

The drone (10) may have mapping capability. The mapping capability may allow the drone (10) to learn a route and repeat this route for future air treatments. If desired, the drone (10) could treat according to a predetermined route and optionally on a set schedule, such as, for example, when the user is sleeping, away from the house or other building to be cleaned. If desired, the drone (10) may be connected to the internet, so that the user remotely knows the treatment cycle has begun or may remotely activate the treatment cycle.

Prophetically, drones (10) suitable for modification to be used with the present invention are made by A.R. Drone of Parrot SA, of Paris, France; under model numbers PF722000 and PF721802B1; by DJI Drones (10) of Shenzhen, China, as Inspire 1 [ASIN: B017MPAPLE], Phantom 2 Vision+[ASIN: BOOPCTNVSC], Phantom 3 [ASIN: B0141PGNSC]; by Blade of Champaign, Ill. under model number BLH8160 [350 QX3]; Guangzhou Walkera Technology Co. Ltd of Guangzhou, China under model number WK-SCOUTX4-RTF2 and IDS Corporation of Pisa, Italy under models numbers IA-17 Manta [fixed wing (12)], SD-150 Hero, IA-12 Stark and IA-3 Colibiri.

For example, the same drone (10), appropriately fitted as described below may be used to clean crumbs from a floor, spray cleaner/other liquids on a target surface, dust a second floor window and clean cobwebs from a third floor ceiling in a single cleaning task. Air treatment may be dispensed from the air treatment dispenser (21) independent of or in conjunction with the cleaning at, for example, any or all of the first, second or third floors.

The drone (10) may have a chassis (14) with one, two, four or more depending struts (18), as is known in the art. A head (20) may be fixedly or pivotally attached to the strut(s) (18) with a single strut (18) in a pedestal configuration being contemplated. If a single strut (18) is used, the head (20) may be mounted to the strut (18) with a fixed connection, a ball and socket joint, universal joint, single axis hinge or other connection. The strut (18) may be telescoping to allow for differential positioning of the head (20) relative to the chassis (14) of the drone (10).

The head (20) may be mounted in a manner similar to that described in U.S. Pat. No. 8,662,438 assigned to Parrot of Paris, France and incorporated herein by reference. The head (20) may have shock absorbers (19) for vertical dampening, to minimize impact and absorb compressive forces between the head (20) and a target surface. The shock absorbers (19) may be mounted between the chassis (14) and the head (20), and particularly may be parallel to or incorporated into the struts (18). Suitable shock absorbers (19) may be hydraulic or pneumatic, as are well known in the art and/or simply may be springs to absorb compressive loads.

### Air Treatment

The air treatment dispenser (21) may be any device suitable for spraying, dripping, coating or otherwise dispensing the air treatment from the drone (10). The air treatment dispenser (21) may comprise an aerosol dispenser, piezo-electric dispenser and/or microfluidic air treatment dispenser (21). Each of these air treatment dispensers (21) may be battery powered and dispense one or more air treatments. Air treatment may be a single, discrete dose, intermittent bursts or continuous.

Particularly, the head (20) may have one or more air treatment dispensers (21) thereon. The air treatment dispenser (21) dispenses air treatments, such as air freshener, autonomously or on demand. The air treatment dispenser (21) may have a refillable and/or replaceable reservoir containing an air treatment. The reservoir

The air treatment may be dispensed in a dispensing direction, which is the centroid of the air treatment as it leaves the air treatment dispenser (21). Particularly, the air treatment may be sprayed in any combination of upwardly, downwardly, laterally and/or into and/or away from one or more rotary wings (12).

The air treatment dispenser (21) may be generally connected to the chassis (14). For example, the air treatment dispenser (21) may be joined to the head (20). The head (20) has a downwardly facing surface which is generally oriented towards ambient. In such an embodiment, the air treatment dispenser (21) may be downwardly oriented to spray away from the wing(s) 12. This arrangement provides the benefit that air treatment is controlled by ambient conditions and may be timed to desired ambient conditions. Without being bound by theory, it is believed that spraying the air treatment form the drone (10) when it is elevated, and particularly into the rotary wings (12) advantageously increases dispersion of the air treatment compared to spraying from a lower elevation or directly to ambient. This arrangement provides the benefit that dispersion of the air treatment is not dependent upon ambient conditions. Prophetically, if the rotating imbalance is accounted for, the air treatment dispenser(s) (21) may be disposed on the rotating wing(s) (12). This arrangement prophetically provides the benefit that maximum dispersion occurs and dispersion can be responsive to elevation of the drone (10).

The drone (10) may comprise plural air treatment dispensers (21). Plural air treatment dispensers (21) may be simultaneously actuated, actuated in series, etc. A first air treatment dispenser (21) may be activated according to demand or to a predetermined schedule. A second, third, fourth air treatment dispenser (21) may be manually actuated by the user according to perceived need or as desired. If plural air treatment dispensers (21) are simultaneously actuated, the air treatment dispensers (21) may dispense the same or different air treatments. Or a first air treatment may be dispensed from a first air treatment dispenser (21), then a second air treatment may be dispensed from a second air treatment dispenser (21), etc.

### Microfluidic Air Treatment

In one embodiment the air treatment dispenser (21) may include a cartridge for a battery powered microfluidic delivery system. The cartridge has a longitudinal axis. The cartridge comprises a reservoir for containing a fluid composition. The cartridge comprises a nozzle operatively connected with the reservoir. The nozzle is in fluid communication with the reservoir for releasing the fluid composition. The cartridge comprises an outer cover operatively connected with the reservoir. The outer cover comprises an orifice that is adjacent to the nozzle. An air flow path is formed by a gap between the reservoir and the outer cover.

In another embodiment, the cartridge may comprise a nozzle operatively connected with the reservoir. The nozzle is in fluid communication with the reservoir for releasing the fluid composition. The cartridge comprises an outer cover operatively connected with the reservoir. The outer cover comprises a top and a skirt that extends from the top of the outer cover and at least partially overlaps with the sidewall of the reservoir along the longitudinal axis. The top of the outer cover comprises an orifice. An air flow path is formed between the outer cover and the reservoir and extends from the skirt to the orifice.

In another embodiment, the cartridge may comprise a reservoir containing a fluid composition to be dispensed from at least one nozzle. The cartridge also comprises an outer cover connected with the reservoir. The outer cover has a top with an orifice disposed adjacent to the nozzle and a skirt extending from the top. At least one of the reservoir and the outer cover may comprise electrical contacts that are electrically connectable with the microfluidic delivery system.

In one embodiment, the microfluidic delivery system may comprise a housing having a base, at least one sidewall connected with the base, and an opening for receiving a cartridge at least partially within the housing. The housing comprises an air inlet. The microfluidic delivery system may comprise a battery powered fan in fluid communication with the housing and a cartridge releasably and electrically connectable with the housing. Or the microfluidic delivery system may rely upon motion of the drone to disperse the fluid composition.

### Sensors

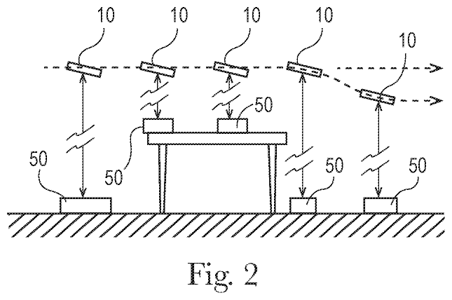

Referring to FIG. 2, the environment in which the drone is used may include commercially available sensors (50) that respond to environmental stimuli such as light, sound, motion, and/or odor levels in the air. For example, the delivery system can be programmed to send a signal to the drone (10) or to the piloting device when the sensor (50) detects light, sound, motion, and/or odor levels in the vicinity of the sensor (50). Upon detection, the drone (10) may autonomously pilot to the vicinity of the sensor (50), and a predetermined region around the sensor (50), to deliver the air treatment. Or the user may receive a signal to his/her phone, laptop, piloting device, etc., and manually pilot the drone (10) to the region of the sensor (50) and deploy the air treatment.

Particularly, one or more VOC sensors (50) can be used to measure intensity of perfume from adjacent or remote devices and alter the operational conditions to work synergistically with other perfume devices. For example a remote sensor (50) could detect distance from the emitting device as well as fragrance intensity and then provide feedback to device on where to locate device to maximize room fill and/or provide the “desired” intensity in the room for the user. The sensors (50) may communicate with each other and coordinate operations in order to work synergistically with other perfume air treatment dispenser (21).

The sensor (50) may also be used to measure fluid composition levels in the reservoir or count firings of associated heating elements to indicate the cartridge's end-of-life in advance of depletion. In such case, an LED light may turn on to indicate the reservoir needs to be filled or replaced with a new reservoir.

The sensors (50) may be in a remote location (i.e. physically separated from the air treatment dispenser (21) such as remote computer or mobile smart device/phone. The sensors (50) may communicate with the delivery system remotely via low energy blue tooth, 6LoWPAN radio, or any other means of wirelessly communicating with a device and/or a controller (e.g. smart phone or computer). The sensors (50) may thereby activate air treatment from the air treatment dispenser (21).

### Fluid Composition

The air treatment may comprise a fluid composition which comprises, consists essentially of or consists of volatile perfume materials and may include a perfume mixture of one or more perfume materials and functional perfume components. The fluid composition may have a viscosity of less than 20 centipoise (“cps”), alternatively less than 18 cps, alternatively less than 16 cps, alternatively from about 5 cps to about 16 cps, alternatively about 8 cps to about 15 cps. And, the volatile composition may have surface tensions from about 20 to about 30 dynes per centimeter as determined using a Bohlin CVO Rheometer system in conjunction with a high sensitivity double gap geometry.

Exemplary volatile materials include perfume materials, volatile dyes, materials that function as insecticides, essential oils or materials that acts to condition, modify, or otherwise modify the environment (e.g. to assist with sleep, wake, antibacterial conditions, respiratory health, etc.), deodorants, cleansers, or malodor control compositions (e.g. odor neutralizing materials such as reactive aldehydes (as disclosed in U.S. 2005/0124512), odor blocking materials, odor masking materials, or sensory modifying materials such as ionones (also disclosed in U.S. 2005/0124512). The air treatment may have an average boiling point of about 70° C. to about 250° C.

### Operation

In operation, the user pilots the drone (10) to the area to be treated, or the drone (10) autonomously pilots on demand or according to a predetermined schedule. The user may manually control the drone (10) throughout the area to be treated, or treatment may be autonomous. The user may repeat areas where additional treatment is needed. Alternatively or additionally the drone (10) may be automatically piloted through a known course or pre-programmed path or piloted using GPS as is known in the art. If desired, the drone (10) may be used outdoors, and piloted through different sites, each having one or more target areas as described in US 2015/0120094 incorporated herein by reference.

Manual control provides the benefit that the user may treat a first target area with the drone (10), then pilot the drone (10) to a second, distant target area, to a third target area, etc. The second target area need not be at the same elevation as the first area. For example, the first target area may be a kitchen and the second target area may be an entryway, separated from the kitchen by steps. Or the first target area may be a table or countertop, and the next target area may be a floor nearby, etc.

Each of these target areas may be lightly treated or more thoroughly treated, depending upon the needs of that particular location. Or different treatments may be customized to the needs of a particular target area. For example, a first target area may be treated with disinfectant, a second target area may be treated with air freshener, a third target area may receive plural treatments, etc.

If desired, the drone (10) may autonomously operate on a predetermined schedule. The drone (10) may travel to designated target areas, at selected times and with equal or unequal frequencies and durations of treatments. This arrangement provides the benefit that the user will not overlook a target area or forget to treat on schedule. Or the drone (10) may respond to a demand signal transmitted from the target area. This arrangement provides the benefit that the treatment occurs only when needed.

The output of the air treatment dispenser (21) may be adjustable or programmable. The rate of fluid output and timing between releases of droplets of fluid composition from the air treatment dispenser (21) may be any desired timing and can be predetermined or adjustable. For example, the air treatment dispenser (21) may be configured to deliver a predetermined amount of the fluid composition, such as perfumes or antibacterials, based on a room size, flight path, proximity to sensors, or may be adjustable by the user. For exemplary purposes only, the flow rate of fluid composition released from the air treatment dispenser (21) maybe about 5 to about 60 mg/hour or any other suitable flow rate. The air treatment dispenser (21) may be used to deliver a fluid composition into the air or be aimed directly towards a surface. Upon depletion of the fluid composition in the reservoir, the microfluidic cartridge may be removed from the housing 10 and replaced with another microfluidic cartridge.

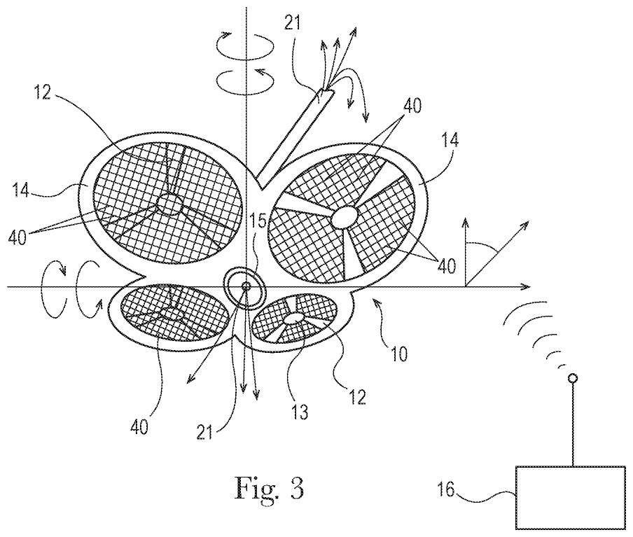

Referring to FIG. 3, the drone (10) may have one or more air treatment filters (40) instead of in addition to the air treatment dispenser (21). The air treatment filters (40) may be disposed on either or both of the intake and/or exhaust of the rotary wing (12), it being generally understood the wings (12) propel air downwardly for lift. This arrangement provides the benefit that additional air treatment modality occurs with normal operation of the drone (10).

The air treatment filters (40) may comprise nonwovens, tow fibers, wadding, cellulose sheets, etc. as are known in the art. The air treatment filters (40) may incorporate perfumes, metal oxide coatings, activated carbon, etc. to clean air passing therethrough.

The air treatment filters (40) may be removably attached to the chassis (14) in known fashion using adhesive, clamps, grippers, and other sheet retainers. This arrangement provides the benefit that the air treatment filters (40) may be easily replaced when clogged or a different type of air treatment filter (40) is needed.

If a quadricopter drone (10) is selected, each rotary wing (12) may have an air treatment filter (40) operatively disposed therewith. This arrangement prevents an imbalance in lift and thrust from occurring if only one, two or three rotary wings (12) have an air treatment filter (40). The air treatment filters (40) may be the same, providing the benefit of matched air flow therethrough. The air treatment filters (40) may be different, providing the benefit of different air treatments occurring in parallel while the drone (10) is in operation.

If desired, such a drone (10) may respond to a signal from a local sensor (50), travel thereto and hover in the vicinity of such sensor (50), passing air through the air treatment filters (40) until the sensor determines the air quality reaches acceptable levels, preventing permeation of the fouled air throughout the environment.

After treatment the drone (10) may piloted so that it returns to the user or a base station. At the base station, the user then replenishes the one or more air treatments in respective reservoirs, as desired, recharges the drone (10), performs maintenance, etc. Or the drone (10) may autonomously pilot directly from a first remote treatment area, to a second remote treatment area, etc.

Optionally, the drone (10) may dispense cleaning fluid instead of or in addition to the air treatment. Such a drone (10) may have a chassis with a depending strut. The strut may have a head with a cleaning sheet removably joined thereto. The drone (10) may be used for wet or dry cleaning in the target area.

### Combinations

A. A drone (10) suitable for treating air, said drone (10) comprising:
a chassis (14) for holding components of the drone (10);
at least one wing (12), the drone (10) being drivable by a respective motor (13);
a central controller for piloting the drone (10); and
a first air treatment dispenser (21) for dispensing an air treatment therefrom, said first air treatment dispenser (21) being connected to said chassis (14).
B. A drone (10) according to paragraph A further comprising a plurality of air treatment dispensers (21), each said air treatment dispenser (21) being adapted to dispense an air treatment therefrom.
C. A drone (10) according to paragraphs A and B comprising a plurality of air treatment dispensers (21), each said air treatment dispenser (21) being adapted to dispense a mutually different air treatment therefrom.
D. A drone (10) according to paragraphs A, B and C comprising a plurality of air treatment dispensers (21), each said air treatment being adapted to dispense mutually different air treatments therefrom in series.
E. A drone (10) according to paragraphs A, B, C and D wherein said air treatment dispenser (21) sprays an air treatment downwardly.
F. A drone (10) according to paragraphs A, B, C, D and E wherein said air treatment dispenser (21) sprays an air treatment upon demand by a user.
G. A drone (10) according to paragraphs A, B, C, D, E and F wherein said central controller is capable of autonomously piloting said drone (10) to an area to be treated and actuating said air treatment dispenser (21) therein according to a predetermined schedule.
H. A drone (10) according to paragraphs A, B, C, D, E, F and G further comprising a second air treatment dispenser (21), said central controller being capable of autonomously piloting said drone (10) to an area to be treated and sequentially actuating said first air treatment dispenser (21), then said second air treatment dispenser (21).
I. A drone (10) according to paragraphs A, B, C, D, E, F, G and H comprising a first air treatment dispenser (21) having a first air treatment, a second air treatment dispenser (21) having a second air treatment, said central controller being capable of autonomously piloting said drone (10) to an area to be treated and sequentially actuating said first air treatment dispenser (21) to dispense a first quantity of said first air treatment, then said second air treatment dispenser (21) to dispense a second quantity of said second air treatment, said first quantity and said second quantity being mutually different.
J. A drone (10) suitable for treating air, said drone (10) comprising:
a chassis (14) for holding components of the drone (10);
at least one rotary wing (12) driven by a respective motor (13);
a central controller for piloting the drone (10); and
an air treatment filter (40) for treating air passing therethrough, said air treatment filter (40) dispensing an air treatment therefrom, said air treatment dispenser (21) being mounted to said chassis (14) and disposed in operative relationship with an intake or an exhaust of said at least one rotary wing (12).
K. A drone (10) according to paragraphs A, B, C, D, E, F, G, H, I and J wherein said drone (10) is a quadricopter having four rotary wings (12), and comprises four air treatment filters (40), each air treatment filter (40) being disposed on a said intake of a respective rotary wing (12).
L. A drone (10) according to paragraphs A, B, C, D, E, F, G, H, I, J and K wherein said drone (10) is a quadricopter having four rotary wings (12), and comprises four air treatment filters (40), each said air treatment filter (40) being disposed on said intake of a respective rotary wing (12), said four air treatment filters (40) comprising at least two mutually different air treatment filters (40).
M. A drone (10) according to paragraphs A, B, C, D, E, F, G, H, I, J, K and L responsive to a signal from an external sensor (50) and which passes air through an air treatment filter (40) upon command from a demand signal.
N. A drone (10) according to paragraphs A, B, C, D, E, F, G, H, I, J, K, L and M further comprising a microfluidic air treatment dispenser (21), said air treatment dispenser (21) being oriented in a respective dispensing direction towards a respective rotary wing (12) to dispense an air treatment from a reservoir.
O. A drone (10) according to paragraph N wherein said reservoir contains an air treatment selected from the group consisting of perfumes, essential oils, reactive aldehydes, ionones and combinations thereof.
P. A drone (10) suitable for treating air, said drone (10) comprising:
a chassis (14) for holding components of the drone (10);
at least one rotary wing (12) driven by a respective motor (13);
a central controller for piloting the drone (10), said central controller being responsive to a demand schedule and/or one or more remote sensors in communication with said central controller, each said remote sensor (50) being associated with a respective remote treatment area; and
an air treatment dispenser (21) for dispensing an air treatment therefrom, said air treatment dispenser (21) being mounted to said chassis (14) and comprising a reservoir for containing an air treatment therein.
Q. A drone (10) according to paragraphs A, B, C, D, E, F, G, H, I, J, K, L M, N, 0 and P wherein said drone (10) is autonomously pilotable to a first remote treatment area upon demand from a sensor (50) therein, and then to a second remote treatment area upon demand from a sensor (50) therein without returning therebetween to a base station.
R. A drone (10) according to paragraphs A, B, C, D, E, F, G, H, I, J, K, L M, N, O, P and Q wherein said drone (10) is autonomously pilotable to a first remote treatment area to dispense a first air treatment therein upon demand from a sensor (50) therein, and then to a second remote treatment area to dispense a second air treatment therein upon demand from a sensor (50) therein, said second air treatment being different from said first air treatment.
S. A drone (10) according to paragraphs A, B, C, D, E, F, G, H, I, J, K, L M, N, O, P, Q and R wherein said drone (10) is autonomously pilotable to a first remote treatment area to dispense a first air treatment therein upon according to a predetermined schedule, and then to a second remote treatment area to dispense a second air treatment therein according to a predetermined schedule.
T. A drone (10) according to paragraphs A, B, C, D, E, F, G, H, I, J, K, L M, N, O, P, Q, R and S wherein said drone (10) is autonomously pilotable to a first remote treatment area to dispense a first air treatment therein from a first elevation, and then to a second remote treatment area to dispense a second air treatment therein from a second elevation, said second elevation being different than said first elevation.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A drone suitable for treating air, said drone comprising:
a chassis for holding components of the drone;
at least one wing, the drone be drivable by a respective motor;
a central controller for piloting the drone; and
a first air treatment dispenser for dispensing an air treatment therefrom, said first air treatment dispenser being connected to said chassis.

2. A drone according to claim 1 further comprising a plurality of air treatment dispensers (21), each said air treatment dispenser being adapted to dispense an air treatment therefrom.

3. A drone according to claim 1 comprising a plurality of air treatment dispensers (21), each said air treatment dispenser being adapted to dispense a mutually different air treatment therefrom.

4. A drone according to claim 1 comprising a plurality of air treatment dispensers (21), each said air treatment being adapted to dispense mutually different air treatments therefrom in series.

5. A drone according to claim 1 wherein said air treatment dispenser sprays an air treatment downwardly.

6. A drone according to claim 1 wherein said air treatment dispenser sprays an air treatment upon demand by a user.

7. A drone according to claim 1 wherein said central controller is capable of autonomously piloting said drone to an area to be treated and actuating said air treatment dispenser therein according to a predetermined schedule.

8. A drone according to claim 1 further comprising a second air treatment dispenser, said central controller being capable of autonomously piloting said drone to an area to be treated and sequentially actuating said first air treatment dispenser, then said second air treatment dispenser.

9. A drone according to claim 1 comprising a first air treatment dispenser having a first air treatment, a second air treatment dispenser having a second air treatment, said central controller being capable of autonomously piloting said drone to an area to be treated and sequentially actuating said first air treatment dispenser to dispense a first quantity of said first air treatment, then said second air treatment dispenser to dispense a second quantity of said second air treatment, said first quantity and said second quantity being mutually different.

10. A drone suitable for treating air, said drone comprising:
a chassis for holding components of the drone;
at least one rotary wing driven by a respective motor;
a central controller for piloting the drone; and
an air treatment filter for treating air passing therethrough, said air treatment filter dispensing an air treatment therefrom, said air treatment dispenser being mounted to said chassis and disposed in operative relationship with an intake or an exhaust of said at least one rotary wing.

11. A drone according to claim 10 wherein said drone is a quadricopter having four rotary wings, and comprises four air treatment filters, each air treatment filter being disposed on a said intake of a respective rotary wing.

12. A drone according to claim 10 wherein said drone is a quadricopter having four rotary wings, and comprises four air treatment filters, each said air treatment filter being disposed on said intake of a respective rotary wing, said four air treatment filters comprising at least two mutually different air treatment filters.

13. A drone according to claim 10 responsive to a signal from an external sensor and which passes air through said air treatment filter upon command from a demand signal.

14. A drone according to claim 10 further comprising a microfluidic air treatment dispenser, said air treatment dispenser being oriented in a respective dispensing direction towards a respective rotary wing to dispense an air treatment from a reservoir.

15. A drone according to claim 14 wherein said reservoir contains an air treatment selected from the group consisting of perfumes, essential oils, reactive aldehydes, ionones and combinations thereof.

16. A drone suitable for treating air, said drone comprising:
a chassis for holding components of the drone;
at least one rotary wing driven by a respective motor;
a central controller for piloting the drone, said central controller being responsive to a demand schedule and/or one or more remote sensors (50) in communication with said central controller, each said remote sensor being associated with a respective remote treatment area; and
an air treatment dispenser for dispensing an air treatment therefrom, said air treatment dispenser being mounted to said chassis and comprising a reservoir for containing an air treatment therein.

17. A drone according to claim 16 wherein said drone is autonomously pilotable to a first remote treatment area upon demand from a sensor therein, and then to a second remote treatment area upon demand from a sensor therein without returning therebetween to a base station.

18. A drone according to claim 16 wherein said drone is autonomously pilotable to a first remote treatment area to dispense a first air treatment therein upon demand from a sensor therein, and then to a second remote treatment area to dispense a second air treatment therein upon demand from a sensor therein, said second air treatment being different from said first air treatment.

19. A drone according to claim 16 wherein said drone is autonomously pilotable to a first remote treatment area to dispense a first air treatment therein upon according to a predetermined schedule, and then to a second remote treatment area to dispense a second air treatment therein according to a predetermined schedule.

20. A drone according to claim 16 wherein said drone is autonomously pilotable to a first remote treatment area to dispense a first air treatment therein from a first elevation, and then to a second remote treatment area to dispense a second air treatment therein from a second elevation, said second elevation being different than said first elevation.
